(19) United States
(12) Patent Application Publication
(10) Pub. No.: US 2017/0016044 A1
(43) Pub. Date: Jan. 19, 2017

(54) TEST STRIP AND APPARATUS FOR MEASURING THE CONTENT OF ALCOHOL IN BLOOD, OR OF ANY OTHER SUBSTANCE IN BLOOD, AND A METHOD FOR MEASURING THE CONTENT OF ALCOHOL IN BLOOD

(71) Applicants: Riikka PYHÄLUOTO, Oulu (FI); Kari KOLEHMAINEN, Kuopio (FI); Ilkka VARTIAINEN, Kuopio (FI)

(72) Inventors: Kari KOLEHMAINEN, Kuopio (FI); IIkka VARTIAINEN, Kuopio (FI); Riikka VARTIAINEN, Oulu (FI)

(73) Assignee: PAL Finland Oy, Kuoplo (FI)

(21) Appl. No.: 15/124,195

(22) PCT Filed: Mar. 12, 2015
(86) PCT No.: PCT/FI2015/050158

§ 371 Date: Sep. 7, 2016

(30) Foreign Application Priority Data
Mar. 13, 2014 (FI) 20145230

(51) Int. Cl. C12Q 1/00; G01N 27/30; G01N 27/327

(52) U.S. Cl. CPC C12Q 1/005; C12Y 101/01001; C12Q 1/004; G01N 27/3272; G01N 27/308; G01N 27/3274; G01N 2333/904

(57) ABSTRACT

A test strip for measuring the blood alcohol content from blood includes a base material and two electrodes attached to it and a reaction area connected to both electrodes, to which area nicotin—amide adenine dinucleotide (NAD⁺) and alcohol dehydrogenase enzyme (ADH) are applied as reagents. A sample opening is connected to the reaction area, from which opening the blood sample to be measured can be transferred to the reaction area to dissolve the reagents and optional auxiliary substances, and at the same time into contact with both electrodes. Between the electrodes can thus be formed a potential difference which makes possible the movement of the protons (H⁺) formed in the reaction towards a negatively charged working electrode, whereby a measurable change in current is formed. The invention further relates to an apparatus and method for measuring the blood alcohol content from blood.

An object of the invention is a test strip for measuring the content of alcohol in blood.

Another object of the invention is an apparatus for measuring the content of alcohol in blood, the apparatus comprising the above-mentioned test strip and a measuring device.

Yet another object of the invention is a method for measuring the content of alcohol in blood.

The apparatus according to the invention and the test applicable to it are any apparatus and test operating on the amperometric principle. Such tests are, for example, glucose (blood sugar), lactate, ethanol (ethyl alcohol), methanol (methyl alcohol), ethylene glycol, and other such tests and analytes and their reagents suitable for measurement by the amperometric principle.

### PRIOR ART

A widely used and known device for determining the blood alcohol content (ethanol content) of a person is an alcometer, by means of which the blood alcohol content is determined from the air exhaled by the person. Alcometers do not, therefore, directly measure the blood alcohol content, which requires a blood test.

The operation of the portable alcometers (“field alcometers”) used by Finnish authorities is based on the electrochemical oxidation of the alcohol contained in the breath. In the work material cell acting as a sensor, alcohol, that is ethanol, breaks down to form acetic acid and an excess of electrons on the top surface of the cell. The electron shortage forming on the lower surface of the cell forms an electric current between the surfaces. The voltage is measured with platinum electrodes and can be read as a per mille reading indicating the ethanol content in the blood.

The currently used alcometers are in principle functional, but they and their use have some disadvantages which are discussed below.

In their field work, paramedics and the police come across people whose blood alcohol content should be measured, but who either refuse to blow into an alcometer or are unable to do so due to their condition.

When paramedics receive a call from the emergency centre, they leave for/arrive at where the patient is. If there is reason to believe that the patient is under the influence of alcohol, a breath test is taken with an alcometer, and the result obtained will determine the treatment to be started. The problem is that the breath test does not reveal the amount of alcohol in the blood. If the patient is unable (unconscious) or unwilling to blow, only primary treatment can be started. The patient has to be transported to a first-aid unit for a blood test to determine the matter.

The weaknesses of the current operations model of the paramedics are, therefore:

a delay in starting treatment

additional costs (extra driving, additional procedures at the first-aid unit).

The alcohol content measurement should, however, be done quickly before further treatment is initiated, because the possible alcohol content in the patient's blood is crucial information for planning further treatment. If the blood alcohol content is not immediately determined correctly, this may lead to a delay in treatment, incorrect procedures and even the loss of a patient's life. A measurement made from the breath does not always give a sufficiently reliable picture of the blood alcohol content.

The police, in turn, use alcometers as screening devices in monitoring the use of intoxicants on the road. If the screening device indicates that there is reason to believe that a driver is guilty of drunken driving, the driver is taken either to a police station for further testing with a precision alcometer or to a health centre for a blood test. If the breath test gives a zero reading although the driver can be identified as an intoxicant user, the driver is taken to a medical examination. Since the ethanol content in the blood does not always correspond to the ethanol content in the breath, the measurements made from the breath may be misleading due to the measuring arrangement. If, on the other hand, a poorly breathing person is in question, a breath test cannot be used for correct assessment of the person's condition even as a starting point.

The weaknesses of the current operations model of the police are, therefore:

additional costs (extra driving, additional procedures at the first-aid unit)

the confirmation done with a precision alcometer, which is expensive to use (precision alcometers are calibrated regularly and the Finnish National Institute for Health and Welfare is responsible for their quality).

Emergency vehicles are often the first to reach a patient and their staff initiates the correct treatment or refer the person into police care. In order to be able to make the correct decision, a clear picture of the ethanol content in the blood is required.

Instead of, or in addition to, an alcometer is, therefore, required a method by means of which the alcohol content of blood can be measured quickly and reliably in field conditions.

### DESCRIPTION OF THE INVENTION

There is, thus, demand for an apparatus by means of which a person's/patient's blood alcohol content can be measured from the blood reliably and accurately in field conditions, regardless of whether the person/patient is conscious or unconscious.

In order to eliminate the above-mentioned weaknesses and to meet the foregoing demand, the test strip according to the invention is characterised in that it relates to a test strip for measuring the blood alcohol content from blood, the said test strip comprising a base material and two electrodes attached to it and a reaction area connected to both electrodes, to which reaction area nicotinamide adenine dinucleotide (NAD⁺) and alcohol dehydrogenase enzyme (ADH) are applied as reagents, and optionally one or more auxiliary substances, a sample opening connected to the reaction area, from which sample opening the blood sample to be measured can be transferred to the reaction area to dissolve the reagents and optional auxiliary substances, and at the same time into contact with both electrodes, between which can be formed a potential difference which makes possible the movement of the protons (H⁺) formed in the reaction towards a negatively charged working electrode, whereby a measurable change in current is formed. According to another aspect of the invention, AOX enzyme (alcohol oxidase enzyme) may alternatively be used instead of ADH.

In a preferred embodiment of the test strip according to the invention, the electrodes are graphite electrodes. To the reaction area of the test strip is preferably applied saccharose and bovine serum albumin (BSA) as auxiliary substances. In another preferred embodiment, the reagents and the possible auxiliary substances are dried to the base, onto the electrodes. The reagents and the possible auxiliary substances are preferably protected with an electro-insulating protective film (e.g. polyethylene film), and that the sample opening penetrates it. The test strip according to the invention preferably includes a temperature sensor.

The apparatus according to the invention is characterised in that it relates to an apparatus for measuring the blood alcohol content from blood, the apparatus comprising a test strip as claimed in any of the claims 1 to 6 and a measuring device with a gate for connecting the test strip to the measuring device, and the operation of which is based on measuring the change in the current (potential difference) between the electrodes in the test strip when the test strip is connected to the gate of the measuring device.

In a preferred embodiment of the apparatus according to the invention, the operating power of the measuring device consists of ordinary batteries, such as three MA batteries. The measuring device according to the invention preferably has a display, for example, an LCD display, which gives the measurement result as mg/l or in per mille.

The method according to the invention is characterised in that includes at least the following stages:

a. a blood test is taken from a person

b. the blood sample is brought through the sample opening of the test strip according to any of the claims 1 to 6 to the reaction area thereof, into contact with the reagents NADH coenzyme and alcohol dehydrogenase enzyme (ADH), whereupon the alcohol (ethanol) possibly contained in the blood sample and the NADH coenzyme react in a reaction catalysed by the alcohol dehydrogenase enzyme (NAD⁺) in such a way that the alcohol (ethanol) is converted into the corresponding aldehyde (acetaldehyde) and proton according to the following reaction equation:

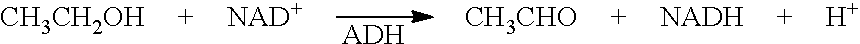

c. the test strip is connected to a device operating on the amperometric principle, which maintains the potential between the two electrodes (1, 2) constant and the instantaneous strength of the current is measured as a function of the protons produced in the reaction.

d. the magnitude of the change in the current caused by the protons produced in the reaction and obtained from the current strength measurement results is converted into a value indicating the alcohol content by means of a computation program.

There has long been a need in the field for the apparatus according to the invention. By means of the invention, this need can now be met. The invention is based on the unexpected observation that the alcohol oxidation reaction catalysed by the alcohol dehydrogenase enzyme (ADH) known as such, wherein ethanol reacts with nicotinamide adenine dinucleotide (NAD⁺), which acts as a coenzyme to form acetaldehyde, a reduced form of nicotinamide adenine dinucleotide (NADH) and protons (H⁺), and may be implemented in the field with a test strip. The reaction equation of this oxidation reaction is:

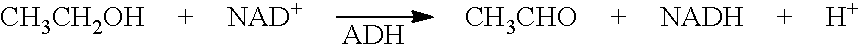

### DETAILED DESCRIPTION OF THE INVENTION

In the following is disclosed in greater detail an example of a preferred embodiment of the strip, apparatus and method according to the invention, with reference to the accompanying drawings, of which

FIG. 1 shows a top view of a preferred embodiment of the test strip according to the invention,

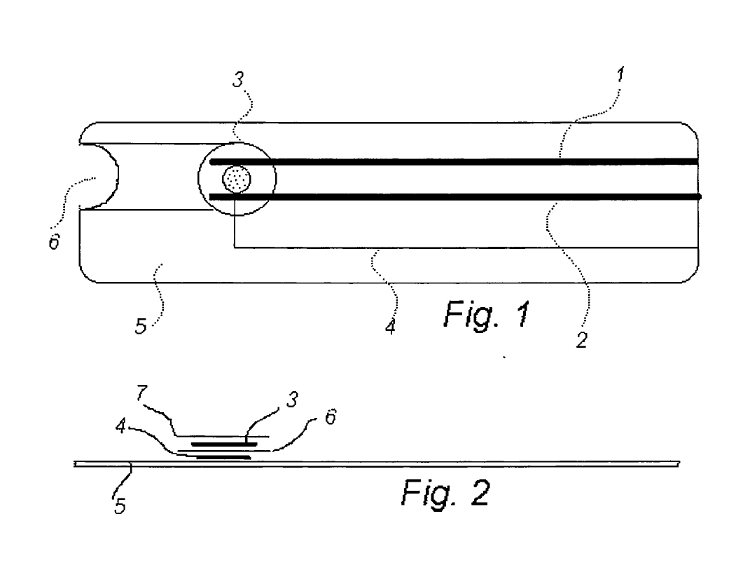

FIG. 2 shows a cross-sectional side view of the test strip shown in FIG. 1,

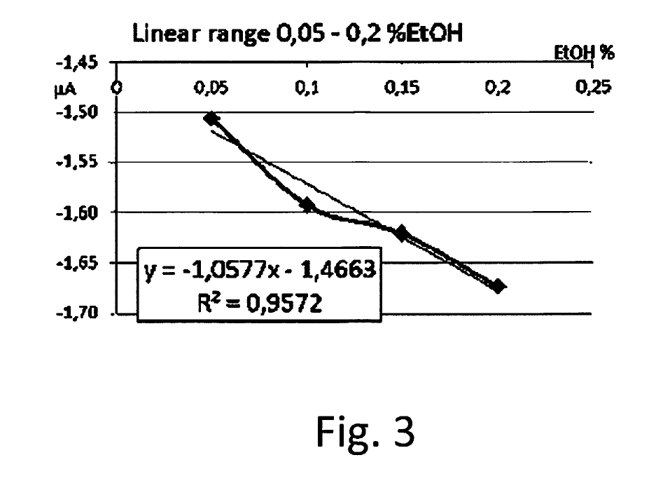

FIG. 3 shows graphically the determination of the blood alcohol content from a blood sample according to a preferred embodiment of the invention.

The invention thus relates to simple and rapid blood alcohol content measurement based on a biosensor. In this preferred embodiment, the apparatus is comprised of a biosensor strip and a measuring device, the operation of which is based on measuring the difference in potential between the graphite electrodes in the biosensor, that is, the test strip, of which one is a working electrode 1 and the other a reference electrode 2. The difference in potential is formed when the ethanol contained in the sample converts to acetaldehyde and a proton (hydrogen, H⁺) in a reaction catalysed by the alcohol dehydrogenase enzyme (ADH). The apparatus contains everything needed for making the measurement. The measurement takes about 3 minutes and can be done without special training. The sample consists of blood from the fingertip, its volume being about 10 μl.

As a measuring device based on potential difference can be used a blood sugar meter applying the glucose oxidase reagent method and operating on the amperometric principle, to which meter is connected an automatic temperature sensor, from which the measuring device receives the corrective reading it uses for calculating the result. One only needs to make experimental measurements at different temperatures and include an algorithm in the program to correct the result to correspond to the room temperature. As an example of a blood sugar meter which, when modified as specified above, can be used to implement the measuring device according to the invention, can be mentioned a blood sugar meter by the name of Mendor Discreet, which is an all-in-one blood sugar meter manufactured and sold by the Finnish company Mendor Oy.

The test strip shown in FIG. 1 comprises the base material 5 and two graphite electrodes 1, 2 attached to it and a reaction area 3 connected to both electrodes. To the reaction area 3 is applied nicotinamide adenine dinucleotide (NAD⁺) and alcohol dehydrogenase enzyme (ADH), and as auxiliary substances saccharose and bovine serum albumin (BSA). The reaction area 3 with its reagents and auxiliary substances is protected outwards with electro-insulating protective film 7 (e.g. polyethylene). The reaction area is connected to a sample opening 6, from which the blood sample to be measured can be transferred to the reaction area to dissolve the reagents and auxiliary substances. At the same time, the blood sample comes into contact with both electrodes 1, 2, between which can, by means of a measuring device operating on the amperometric principle, be formed a potential difference, which enables the protons (H⁺) formed in the reaction to move towards the negatively charged working electrode 1, whereby a measurable change in current is formed.

The reagents and auxiliary substances in the test strip have been added and dried onto the electrodes 1, 2. The addition of reagents and auxiliary substances to the base 5, onto the electrodes, takes place by means of industrial liquid metering, after which the reagents and auxiliary substances are dried, and finally protected with an insulating protective film 7, such as a polyethylene film.

As can be seen in FIG. 2, the test strip is in general comprised of graphite electrodes 1, 2 placed on solid material, between which is formed a reaction area 3 to which the reagents and auxiliary substances have been applied in liquid form and then dried. In addition to these, the test strip also comprises a temperature sensor 4, which is positioned under the reaction area 3, separated by an insulator 6. The temperature sensor 4 is thus fixed directly to the base 5, on top of which the insulator 6 is mounted to insulate the sensor from the reaction area.

The blood alcohol content is measured as follows:

a. a blood sample is taken, for example, from a person's fingertip

b. the blood sample is brought through the sample opening of the test strip (by bringing the drop of blood into contact with the sample opening, it is absorbed in the strip) to the test strip reaction area, into contact with the reagents NADH coenzyme and alcohol dehydrogenase enzyme (ADH), whereupon the alcohol (ethanol) possibly contained in the blood sample and the NADH coenzyme react in a reaction catalysed by the alcohol dehydrogenase enzyme (NAD⁺) in such a way that the alcohol (ethanol) is converted into the corresponding aldehyde (acetaldehyde) and a proton according to the following reaction equation:

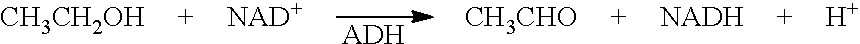

c. the test strip is connected to a device operating on the amperometric principle, which maintains the potential between the two electrodes (1, 2) constant and the instantaneous strength of the current is measured as a function of the protons produced in the reaction.

d. the magnitude of the change in the current caused by the protons produced in the reaction and obtained from the current strength measurement results is converted by means of a computation program into a value indicating alcohol content.

The measuring device operating according to the amperometric principle is a class I IVD medical device and intended for professional use. The operating power consists of three AAA batteries. The measuring device and the apparatus as a whole do not present an immediate risk to the user or patient due to the conduction of power, nor a biological risk. The apparatus includes software for controlling the use and monitoring of the apparatus. The accessories include a calibrator, control and lancets needed for taking the sample. Taking the sample requires special know-how, which must be included in the device's user requirements. The proper functioning of the device requires a temperature of 10-30° C. and a relative humidity of 10-80%. For longer storage periods, the device package should be kept at room temperature (10-25° C.). The shelf life of the strips is at least six months at the appropriate temperature. The measurement is done directly from the fingertip blood sample; transporting the sample elsewhere is not recommended. The measuring accuracy within the range from 0.5-6.0% is less than ±20 CV-%.

The properties of the measuring device

size 100×50×30 mm

weight 100-200 g

product package

device, strips, instructions for use

implements required for sampling

bag carried on belt

LCD display

result in mg/l and per mille

date

strip indicator

user interface

2 push buttons

menu and acknowledgement functions

operating power 3×AAA batteries

sensor gate

sensor identification

developing the measuring device does not require special know-how

an abundance of manufacturers available

manufacturing costs in large production runs approximately 20-30

[symbol]

device comprises internal calibration; external calibration is carried out with standard material

several versions can be made of the device, e.g. equipped with data transfer

User sectors for the apparatus:

First aid/ambulances

Police in traffic control

First-aid units of hospitals and health centres

Consumer markets

Other random users (e.g. some employers, schools, the army, etc.)

FIG. 3 shows graphically the determination of the blood alcohol content from a (blood) sample according to a preferred embodiment of the invention, by using an alcohol oxidase (AOX)-Prussian blue-transmitted graphite sensor. The Figure shows the linear ratio between the alcohol (ethanol, EtOH) content and the amperometric current change by means of the so-called constant descriptor equation y=−1.0577x−1.4663 (the square of the equation's coefficient R²=0.9572). By means of the equation in the memory of the measuring device according to the invention can reliably be calculated the ethanol content in the blood sample of the person/patient being examined.

1. A test strip for measuring the blood alcohol content from blood, the test strip comprising a base material and two electrodes, attached to it and a reaction area connected to both electrodes, to which reaction area nicotinamide adenine dinucleotide (NAD⁺) and alcohol dehydrogenase enzyme (ADH) are applied as reagents, and optionally one or more auxiliary substances, a sample opening in connection with the reaction area, from which sample opening the blood sample to be measured can be transferred to the reaction area to dissolve the reagents and optional auxiliary substances, and at the same time into contact with both electrodes, between which can be formed a potential difference which makes possible the movement of the protons (H⁺) formed in the reaction towards a negatively charged working electrode, whereby a measurable change in current is formed.

2. The test strip as claimed in claim 1, wherein the electrodes are graphite electrodes.

3. The test strip as claimed in claim 1, to the reaction area of which saccharose and bovine serum albumin (BSA) are applied as auxiliary substances.

4. The test strip as claimed in claim 1, wherein the reagents and possible auxiliary substances are dried to the base, onto the electrodes.

5. A The test strip as claimed in claim 1, wherein the reagents and possible auxiliary substances are protected with an electro-insulating protective film (e.g. polyethylene film), and which the sample opening penetrates.

6. The test strip as claimed in claim 1, which comprises a temperature sensor.

7. An apparatus for measuring the blood alcohol content from blood, the apparatus comprising a test strip as claimed in claim 1 and a measuring device with a gate for connecting the test strip to the measuring device, and the operation of which is based on measuring the change in the current (potential difference) between the electrodes in the test strip when the test strip is connected to the gate of the measuring device.

8. The apparatus as claimed in claim 7, wherein the operating power of the measuring device consists of ordinary batteries, for example, three AAA batteries.

9. The apparatus as claimed in claim 7, wherein the measuring device has a display, for example, an LCD display, which gives the measurement result as mg/l or in per mille.

10. A method for measuring the blood alcohol content, comprising at least the following steps:
a. a blood sample is taken from a person
b. the blood sample is brought through the sample opening of the test strip according to to the reaction area thereof, into contact with the reagents NADH coenzyme and alcohol dehydrogenase enzyme (ADH), whereupon the alcohol (ethanol) possibly contained in the blood sample and the NADH coenzyme react in a reaction catalysed by the alcohol dehydrogenase enzyme (NAD⁺) in such a way that the alcohol (ethanol) is converted into the corresponding aldehyde (acetaldehyde) and a proton according to the following reaction equation: 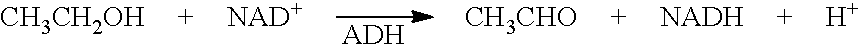
c. the test strip is connected to a device operating on the amperometric principle, which maintains the potential between two electrodes constant and the instantaneous strength of the current is measured as a function of the protons produced in the reaction; and
d. the magnitude of the change in the current caused by the protons produced in the reaction and obtained from the current strength measurement results in converted by means of a computation program into a value indicating alcohol content.

11. A test strip for measuring the content of a substance (analyte) in blood from the blood, the test strip comprising a base material and two electrodes attached to it and a reaction area connected to both electrodes, to which is (are) applied a reagent(s), which reacts (react) with the substance contained in the said blood in a reaction which releases protons (H⁺), and optionally one or more auxiliary substances, a sample opening (6) in connection with the reaction area, from which sample opening the blood sample to be measured can be transferred to the reaction area to dissolve the reagents and optional auxiliary substances, and at the same time into contact with both electrodes, between which can be formed a potential difference which makes possible the movement of the protons (H⁺) formed in the reaction towards a negatively charged working electrode, whereby a measurable change in current is formed.

12. The test strip as claimed in claim 11, wherein the substance contained in the blood, the content which in the blood is measured, is glucose (blood sugar), lactate, ethanol (ethyl alcohol), methanol (methyl alcohol), or ethylene glycol.

13. The test strip as claimed in claim 12, wherein the substance contained in the blood, the content of which in the blood is measured, is ethanol.

14. The test strip as claimed in claim 13, wherein the reagents used for measuring the ethanol content are nicotinamide adenine dinucleotide (NAD⁺) and alcohol dehydrogenase enzyme (ADH) or an alternative oxidase (alcohol oxidase (AOX)).

15. The test strip as claimed claim 11 in, wherein the electrodes are graphite or graphene electrodes.

16. A The test strip as claimed in claim 11, to the reaction area of which is applied saccharose and bovine serum albumin (BSA) as auxiliary substances.

17. The test strip as claimed in claim 11, wherein the reagents and possible auxiliary substances are dried to the base, onto the electrodes.

18. The test strip as claimed in claim 11, wherein the reagents and possible auxiliary substances are protected with an electro-insulating protective film (e.g. polyethylene film), and which the sample opening penetrates.

19. The test strip as claimed in claim 11, further comprising a temperature sensor, which automatically feeds a corrective value corresponding to the temperature at the time of measurement to the measuring device, when the test strip is connected to the measuring device, there being an algorithm in the program in the measuring device, which corrects on the basis of the said temperature correction value, the temperature value measured by the temperature sensor to correspond to room temperature, whereby temperature variations at the time of measurement cannot impair (distort) the measurement result.

20. An apparatus for measuring the content of a substance (analyte) in blood from the blood, the apparatus comprising a test strip as claimed in claim 11 and a measuring device which comprises a gate for connecting the test strip to the measuring device, and the operation of which is based on measuring the change in the current (potential difference) between the electrodes in the test strip, when the test strip is connected to the gate of the measuring device.

21. The apparatus as claimed in claim 20, wherein operating power of the measuring device consists of ordinary batteries, for example, three AAA batteries.

22. The apparatus as claimed in claim 20, wherein the measuring device comprises a display, which gives the measurement results as mg/l or in per mille.
